(19) United States
(12) Patent Application Publication
(10) Pub. No.: US 2024/0325457 A1
(43) Pub. Date: Oct. 3, 2024

(54) PLACENTAL TISSUE PARTICULATE COMPOSITIONS AND METHODS OF USE

(71) Applicants: Doron Therapeutics, Inc., Chapel Hill, NC (US); Musculoskeletal Transplant Foundation, Edison, NJ (US)

(72) Inventors: Alessandra PAVESIO, Durham, NC (US); Carl FLANNERY, Durham, NC (US); Scott SEAMAN, Durham, NC (US); Michael A. NASERT, Hazlet, NJ (US); Eric J. SEMLER, Morganville, NJ (US); Marc LONG, Denville, NJ (US)

(21) Appl. No.: 18/608,371

(22) Filed: Mar. 18, 2024

Related U.S. Application Data
(62) Division of application No. 17/097,350, filed on Nov. 13, 2020.
(60) Provisional application No. 62/938,472, filed on Nov. 21, 2019.

(51) Int. Cl. A61K 35/50; A61K 9/00; A61K 35/51; A61K 38/17; A61K 38/18; A61K 38/20; A61K 38/39; A61K 38/57; A61P 19/02

(52) U.S. Cl. CPC A61K 35/50; A61K 9/0019; A61K 35/51; A61K 38/1793; A61K 38/18; A61K 38/1825; A61K 38/2006; A61K 38/39; A61K 38/57; A61P 19/02

(57) ABSTRACT

Provided herein are compositions containing dehydrated placental tissue particulates, methods of making the compositions and methods for treating various musculoskeletal disorders and other conditions using such compositions, including osteoarthritis (OA), degenerative disc disease, tendonitis, plantar fasciitis, and pain associated therewith.

### CROSS-REFERENCE TO RELATED APPLICATIONS

This application is a divisional of U.S. application Ser. No. 17/097,350, filed Nov. 13, 2020, the contents of which are hereby incorporated herein by reference. U.S. application Ser. No. 17/097,350, claims priority to U.S. Provisional Patent Application Ser. No. 62/938,472, filed on Nov. 21, 2019, entitled “PLACENTAL TISSUE PARTICULATE COMPOSITIONS AND METHODS OF USE” the content of which is incorporated herein by reference.

### FIELD OF THE INVENTION

The invention relates generally to the field of pharmaceuticals and medical treatments including allogeneic tissue grafts and methods of making and using the same. More particularly, the invention relates to compositions containing dehydrated placental tissue particulates, methods of making of such compositions and methods for treating various musculoskeletal disorders and other conditions using such compositions, including osteoarthritis (OA), degenerative disc disease, tendonitis, plantar fasciitis, and pain associated therewith.

### BACKGROUND OF THE INVENTION

The placenta, which surrounds the fetus during gestation, is composed of several tissue types. The umbilical cord connects the placenta to the fetus, and transports oxygen to the fetus. The outer “shell” of the placenta is known as the “chorion” which functions to protect and nurture the embryo. The chorionic fluid prwotects the embryo from shock, and the chorionic villi, which are extensions of the chorionic villous tree, allow the exchange of nutrients, oxygen and waste products with the mother. The amniotic membrane (AM) is the innermost layer of the placenta tissue closest to the fetus, separating the mother from the fetus throughout the baby's developmentis. The AM forms an avascular membranous sac that is filled with amniotic fluid and comprises an intermediate layer, an epithelial layer and a subadjacent avascular stromal layer.

Human placental tissue preparations have been used in medicine for over 100 years and are now used to treat difficult-to-heal wounds and soft tissue injuries. Umbilical cord tissue is also currently used in the field of regenerative medicine to treat injuries and chronic, degenerative conditions. The use of micronized placental tissue particles has provided benefits to disease and injured tissue. However, the processing, formulation and clinical usage of micronized placental tissue particles have not been optimized to fully meet the needs of patients in alleviating pain and treating other symptoms of musculoskeletal disorders and other medical conditions. Thus, there is a need for improved placental tissue compositions for use in the medical and surgical fields.

### SUMMARY OF THE INVENTION

Described herein are, dehydrated placental tissue particulate (PTP) compositions, kits and methods of use in medical treatment of various musculoskeletal disorders and other medical conditions. Also described herein are methods for preparing the PTP compositions and preparations. Also described are methods for using the PTP compositions and preparations for medical treatment, including prophylactic methods.

In one aspect of the invention, there are provided PTP compositions that are prepared from amniotic membrane (AM), chorion membrane (CM) and umbilical cord (UC) obtained from human placenta. The PTP compositions comprise a mixture of dehydrated placental tissue particulates (PTP) comprising from about 10 wt % to about 30 wt % amnion particulates, about 30 wt % to about 75 wt % chorion particulates and about 5 wt % to about 50 wt % umbilical cord particulates. In certain embodiments of this aspect of the invention, the placental tissue particulates have a particle size in the range of from 20 to 150 microns. In certain embodiments, the AM, CM and UC particulates are obtained from mammalian placental tissue. In certain embodiments, the composition can be utilized in a dry format or as a suspension comprising a minimum of 10 mg/mL of said PTP. In embodiments of this aspect, the compositions may comprise at least about 40 mg, 50 mg, 60 mg, 70 mg, 80 mg, 90 mg, 100 mg or more of a mixture of placental tissue particulates (PTP), said PTP comprising about 10 wt % to about 30 wt % AM particulates, about 30 wt % to about 75 wt % CM particulates and about 5 wt % to about 50 wt % UCC particulates. In embodiments of this aspect, the PTP compositions comprise quantifiable amounts of each of basic fibroblast growth factor (bFGF), interleukin-1 receptor antagonist (IL-1Ra), interleukin-1alpha (IL-la), tissue inhibitor of metalloproteases (TIMP)-1, TIMP-2, TIMP-3, and fibronectin.

In another aspect of the invention, there is provided a method of treating a musculoskeletal disorder or orthopedic condition in a subject in need thereof, comprising administering to the subject one or more doses of a composition comprising about 50-300 mg of a mixture of placental tissue particulates (PTP), such as 100-200 mg. The PTP compositions comprise about 10 wt % to about 30 wt % AM particulates, about 30 wt % to about 75 wt % CM particulates and about 5 wt % to about 50 wt % UC particulates, wherein when multiple doses of the composition are administered, a first and second dose of said multiple doses are administered at least one to two weeks apart. In embodiments of this aspect, the PTP compositions contain quantifiable amounts of each of bFGF, IL-1Ra, IL-1α, TIMP-1, TIMP-2, TIMP-3, and fibronectin. In certain embodiments, the musculoskeletal disorder or orthopedic condition is selected from the group consisting of osteoarthritis, degenerative disc disease, cartilage deficits or damage, soft tissue injury, physical trauma and orthopedic surgery. In certain embodiments of this aspect, one or more doses of the PTP composition is administered by localized injection of the resuspended PTP composition. In other embodiments, one or more doses of the PTP composition is administered by application of the dehydrated PTP to the target site. In certain embodiments, the method results in reduced pain associated with the musculoskeletal disorder and in yet other embodiments, the method results in reduction or inhibition of cartilage degeneration and/or bone damage associated with the musculoskeletal disorder or orthopedic condition.

In yet another aspect of the invention, there is provided a method of treating pain associated with osteoarthritis in a subject in need thereof, comprising administering to a site of osteoarthritis pain a composition comprising at least about 50 mg of a mixture of resuspended dehydrated placental tissue particulates (PTP), said PTP comprising about 10 wt % to about 30 wt % AM particulates, about 30 wt % to about 75 wt % CM particulates and about 5 wt % to about 50 wt % UC particulates. In certain embodiments, a single dose of the PTP is administered and in other embodiments, one or more additional doses of the PTP composition are administered starting at least one week after administering the first dose of the PTP composition. In embodiments of this aspect, the PTP composition comprises quantifiable amounts of each of bFGF, IL-1Ra, IL-1α, TIMP-1, TIMP-2, TIMP-3, and fibronectin.

In another aspect of the invention, there is provided a kit comprising one or more doses of a pharmaceutical composition comprising a therapeutically effective amount of a mixture of lyophilized placental tissue particulates (PTP), said PTP comprising from about 10 wt % to about 30 wt % amnion particulates, about 30 wt % to about 75 wt % chorion particulates and about 5 wt % to about 50 wt % umbilical cord particulates. In embodiments of this aspect, the PTP composition comprises quantifiable amounts of each of bFGF, IL-1Ra, IL-la, TIMP-1, TIMP-2, TIMP-3, and fibronectin. In certain embodiments, the kits further comprising a pharmaceutically acceptable excipient. In certain embodiments, the kits comprise instructions for administering the one or more doses.

In another aspect of the invention, there is provided a method for preparing a pharmaceutical composition for point of care medical treatment, comprising the steps of (1) separating AM, CM and UC from placental tissue, (2) cutting the AM, CM and UC into multiple pieces to obtain separate AM, CM and UC tissue pieces and lyophilizing the pieces; (3) cryomilling the AM, CM and UC tissue pieces separately to obtain milled AM, CM and UC tissue to obtain particulates having a size of 20 to 150 microns; (4) combining a predetermined amount of each of the AM, CM and UC milled tissue to obtain a mixture comprising from about 10 wt % to about 30 wt % AM particulates, about 30 wt % to about 75 wt % CM particulates and about 5 wt % to about 50 wt % UC particulates; (5) lyophilizing the mixture; and optionally (6) sterilizing the particulates of step (5). In certain embodiments, the method for preparing the pharmaceutical composition further comprises the step of reconstituting the particulates in a sufficient amount of a sterile aqueous solution to form a suspension of the particulates. In embodiments of this aspect, the PTP composition comprises quantifiable amounts of each of bFGF, IL-1Ra, IL-1α, TIMP-1, TIMP-2, TIMP-3, and fibronectin.

In a further aspect of the invention, there is provided a PTP composition made by a process comprising the steps of:
(1) separating AM, CM and UC from placental tissue,
(2) cutting the AM, CM and UC into multiple pieces to obtain separate AM, CM and UC tissue pieces and lyophilizing the pieces;
(3) cryomilling the AM, CM and UC tissue pieces separately to obtain milled AM, CM and UC particulates having a particle size of 20 to 150 microns;
(4) combining a predetermined amount of each of the AM, CM and UC milled particulates to obtain a mixture comprising from about 10 wt % to about 30 wt % AM particulates, about 30 wt % to about 75 wt % CM particulates and about 5 wt % to about 50 wt % UC particulates;
(5) lyophilizing the mixture; and optionally
(6) sterilizing the particulates of step (5),
   wherein the PTP composition comprises quantifiable amounts of each of bf growth factor (bFGF), IL-1Ra, IL-1α, TIMP-1, TIMP-2, TIMP-3, and fibronectin.

All publications mentioned herein are incorporated by reference in their entirety. In the case of conflict, the present specification, including definitions will control. In addition, the particular embodiments discussed below are illustrative only and not intended to be limiting.

### Definitions

The term “therapeutically acceptable” with respect to a formulation, composition or component, as used herein, means having no persistent detrimental effect on the general health of the subject being treated.

The term “therapeutically effective amount,” as used herein, refers to a sufficient amount of an agent or a compound or composition being administered which will relieve, partially or fully, one or more of the symptoms of the disease or condition being treated, e.g., tissue damage or associated pain or other symptoms or causes of the treated disease.

The term “pharmaceutically acceptable,” as used herein, refers to a material which is relatively nontoxic, i.e., the material may be administered to an individual without causing undue undesirable biological effects or interacting in a deleterious manner with any of the components of the composition in which it is contained.

The term “placental tissue” as used herein refers to components of placental tissue including whole placental tissue that has been modified by cleaning and segmenting the various tissues as well as to separate amnion membrane, chorion membrane, and umbilical cord. Placental tissue may contain extracellular matrix layers naturally found in the placenta, such as epithelial layers, fibroblast layers, an intermediate layer, a trophoblast layer, Wharton's jelly and the like.

The terms “lyophilized” and “dehydrated” are used interchangeably herein to mean the state, but not the method, of having had water removed as a means of preservation.

The terms “resuspend” or “resuspended are used herein to refer to the addition of a liquid, e.g., a diluent to a dehydrated material in order to suspend the material in a solution and to allow for injection through an adequately sized needle.

The term “diluent” refers to chemical compounds that are used to dilute, suspend or resuspend the compound or composition of interest prior to delivery. Salts dissolved in buffered solutions (which also can provide pH control or maintenance) are utilized as diluents in the art, including, but not limited to a phosphate buffered saline solution and sodium chloride solutions.

The term “point of care” is used herein to mean at or near the point in time when a clinician or other health care provider administers health care services and/or products, including the composition of the invention, to a patient.

The term “about”, as used herein, encompasses the explicitly recited amounts as well as deviations therefrom of +5%.

### BRIEF DESCRIPTION OF THE DRAWINGS

FIG. 1 is a bar graph showing the results of protein multiplex analyses performed on PTP samples using a custom Quantibody® Array to assess for the presence of specific pro-anabolic, inflammatory and anti-catabolic/anti-inflammatory growth factors and cytokines. N=1 donor.

FIG. 2 is a bar graph showing representative results from ELISA testing of PTP for therapeutically relevant growth factors, cytokines, and matrix proteins. bFGF, basic fibroblast growth factor; IL-1Ra, interleukin-1 receptor antagonist; IL-1α, interleukin-1 alpha; TIMP-1,-2, -3, tissue inhibitor of metalloprotease-1,-2,-3; and fibronectin (Fn).

FIG. 3 is a representative MMP-13 Western blot from primary human OA articular chondrocyte characterization studies of PTP.

FIG. 4 is a graph showing PTP dose-dependent inhibition of inflammation-induced MMP-13 by primary human OA articular chondrocytes, using PTP from 2 individual placental tissue donors (designated as donor 8 and donor 9).

FIG. 5 is a bar graph showing representative results obtained from the destabilization of the medial meniscus (DMM) surgical model of OA with single dose administration of PTP compositions of the invention and hyaluronic acid (HA) and steroid on weight-bearing pain over six weeks.

FIG. 6 is a bar graph showing representative results obtained from the DMM surgical model of OA with single dose administration of PTP compositions of the invention and hyaluronic acid (HA) and steroid on von Frey pain over six weeks.

FIG. 7 is a bar graph showing representative results obtained from the DMM surgical model of OA with dual dose administration of PTP compositions of the invention at two concentrations on weight-bearing pain and von Frey pain over six weeks.

FIGS. 8A and 8B are bar graphs showing the results obtained from the DMM surgical OA model for Area Under the Curve (AUC) cumulative pain values over 6 weeks for weight-bearing pain (FIG. 6A, and for Von Frey pain (FIG. 6B).

FIG. 9 is a series of bar graphs showing the histology results obtained at 4 weeks post-second dose from a rat surgical model of OA in animals dosed twice with saline or PTP.

FIG. 10 is a series of histology slides obtained from a rat model of OA in animals dosed twice with saline or high dose PTP.

FIG. 11 is a bar graph showing representative results obtained from the collagenase-induced model of tendinitis with single dose administration of a PTP compositions of the invention and steroid on weight-bearing pain over 30 days.

FIG. 12 is a bar graph showing representative results obtained from the collagenase-induced model of tendinitis with single dose administration of a PTP composition of the invention and steroid on von Frey pain over six weeks.

FIG. 13 is a series of bar graphs showing the histology results obtained at 30 days post dosing from the collagenase-induced model of tendinitis in animals dosed with saline or PTP or steroid.

FIG. 14 is a series of histology slides obtained from the collagenase-induced model of tendinitis in animals dosed with saline or PTP or steroid.

FIG. 15 is a series of bar graphs showing representative levels of some of the growth factors, cytokines and ECM proteins contained within UC, AM and CM tissue.

FIG. 16 is a bar graph showing the results of protein multiplex analyses performed on PTP samples using a custom Quantibody® Array to assess for the presence of specific pro-anabolic, inflammatory and anti-catabolic/anti-inflammatory growth factors and cytokines. N=7 donors, data: mean+SEM.

FIG. 17 is a bar graph showing representative results from external, validated ELISA testing of PTP for therapeutically relevant growth factors, cytokines, and matrix proteins. bFGF, basic fibroblast growth factor; IL-1Ra, interleukin-1 receptor antagonist; IL-1α, interleukin-1 alpha; TIMP-1,-2,-3, tissue inhibitor of metalloprotease-1,-2,-3; and fibronectin (Fn). N=12 donors, data: mean+SD.

### DETAILED DESCRIPTION OF THE INVENTION

Described herein are dehydrated placental tissue particulate (PTP) compositions that may be re-constituted, at point of care for example, for treatment of various disease states and medical conditions. The PTP compositions exert a number of physiologically significant effects in mammalian cells and intact mammalian tissues, rendering the compositions particularly useful in the treatment of musculoskeletal disorders. For example, injectable formulations of the PTP compositions may be used for the treatment of osteoarthritis, degenerative disc disease, tendonitis, cartilage deficits or damage, trauma, plantar fasciitis, soft tissue damage and other musculoskeletal disorders.

The dehydrated PTP compositions described herein comprise at least three isolated placenta tissue components: umbilical cord (UC), amnion membrane (AM) and chorion membrane (CM). Each of the components may be obtained from a single donor placenta and processed separately. However, it is also possible to obtain the placenta tissues from various individual placentas and/or process the isolated tissues in a batch process.

Additional components may also be included in PTP compositions containing UC, AM and CM. Non-limiting examples of such additional components include for example, hyaluronic acid (HA) and HA derivatives, antibiotics, anti-inflammatoires, corticosteroids, surfactants, anti-caking agents, and the like.

Each of the three placental tissue components of the PTP compositions described herein is prepared from mammalian placenta, preferably human placenta and more preferably, human placental tissue delivered by Cesarean section, although human placenta delivered by natural childbirth may also be used. The placenta may be processed immediately after recovery or stored at about 2° to 8° C. for example for up to 90 hours post-recovery, preferably no longer than 72 hours post-recovery. The inventors have found that freshly obtained placenta (stored no longer than 90 hours at 2° to 8° C., for example, preferably no longer than 72 hours post-recovery) limits the risk of degeneration of the tissues. However, frozen AM, CM and/or UC tissue, in place of fresh tissue or in combination with fresh tissue may also be used.

In another aspect of the invention, there is provided a method for preparing the PTP compositions described herein. The process described herein provides a PTP composition having particles sized appropriately for delivery via syringe and provides processing procedures that do not adversely affect growth factors and cytokines of interest which are intrinsic to the placental tissue. The entire procedure for preparing PTP compositions may be performed aseptically. In preparing the placenta particulates, the UC is detached from the placenta and CM and AM are separately removed. Blood clots from the AM and CM are removed if present and blood vessels are removed from the UC. In one embodiment, all extracellular layers of the amnion, chorion and umbilical cord present in native tissue are retained during processing. In alternative embodiments, one or more or more layers (for example but not limited to, epithelial layer, spongy layer, trophoblast layer, Wharton's Jelly, etc.) are removed. Preferably, the tissues are rinsed one or more times, for example in Phosphate Buffered Saline (PBS) to remove blood and other contaminants. Preferably, but not necessarily, the three tissue types are rinsed separately. By rinsing the tissues as separate components, contaminants and blood are more easily and thoroughly removed. Rinsing can be done at room temperature or under cooling using PBS or other suitable aqueous solution. Chemical disinfection of the tissues is not necessary to reduce bioburden and carries the risk of reduced biofactor content and/or activity. Viral inactivation can optionally be carried out. In one embodiment, the tissues may be soaked in a solution during processing that can provide viral inactivation or viral load reduction. Examples of such solutions include detergents, alcohols, acids, bases or combinations thereof. In another embodiment, the tissues may be exposed to elevated or reduced temperatures in order to provide viral inactivation.

The three tissue types are each cut into smaller pieces, e.g., 4 cm strips for further processing. The pieces are dehydrated, e.g., lyophilized for about 12 to 20 hours, such as 18 hours or using other method of dehydration. Following dehydration, the pieces are cryogenically or freezer milled, preferably with each tissue type milled separately. A cryogenic or other freezer milling procedure can be utilized whereby the placental tissues are pulverized while frozen. This ensures that an appropriate particle size can be generated without subjecting the tissue to conditions that might negatively affect the endogenous proteins. As used herein, the terms “cryogenic,” or “cryogenically” in reference to a milling process means the tissues are milled in a cryogen (such as liquid nitrogen or liquid argon) slurry or at a cryogenic temperature under processing parameters. “Freezer milling” is a type of cryogenic milling that is used to mill samples usually at liquid nitrogen temperatures. The terms “cryogenically milled” and “freezer milled” are used interchangeably herein to indicate that the tissue was pulverized under freezing conditions that resulted in the appropriately sized particles while maintaining the integrity of the biological materials being processed. Any milling process that pulverizes the placental tissues to an appropriate particle size while maintaining the tissues in a frozen state while milling may be used in the present invention.

The cryogenic/freezer milled tissues are preferably weighed to determine the amount of each tissue. If milled separately, the milled tissues are recombined for further processing. A predetermined amount of each tissue is combined to formulate a PTP composition having a desired weight percentage of each tissue type. The milled particles are separated by size, for example by centrifugation or sedimentation or by passing through a sieve to provide a particle size of the mixed components of about 5 to 300 microns, preferably about 10 to 200 microns or more preferably 20 to 150 microns. One of skill in the art will appreciate that when particles are separated using a sieve, a dimension of at least a portion of the particles may be larger than the opening of the sieve used due to the shape of the particles. The size of the placental tissue particulates may be selected to ensure that a suspension of the particulates suitable for injection through a syringe may be obtained. Preferably, the milled particulates are extrudable through a 22 gauge needle or higher, a 25 gauge needle or higher, or a 27 gauge needle or higher when in suspension in liquid.

Once size separation is complete, the milled tissue mixture may be aliquoted by weight and optionally may be dehydrated a final time, e.g., lyophilized for 12 to 20 hours, such as 18 hours. The residual moisture content of the final dehydrated or lyophilized PTP is preferably less than 15%, more preferably less than 6%.

The aliquots of lyophilized or otherwise dehydrated PTP may be distributed in individual containers such as glass vials that are stoppered under vacuum and capped to create a hermetic seal, for example. The aliquoted, lyophilized tissue is optionally sterilized by E-Beam irradiation, gamma irradiation, UV light or exposure to ethylene oxide, supercritical carbon dioxide or other suitable sterilant known to those in the art. E-beam irradiation is a preferred method of sterilization. The dehydrated, sterilized PTP compositions are substantially free of blood residuals and foreign matter.

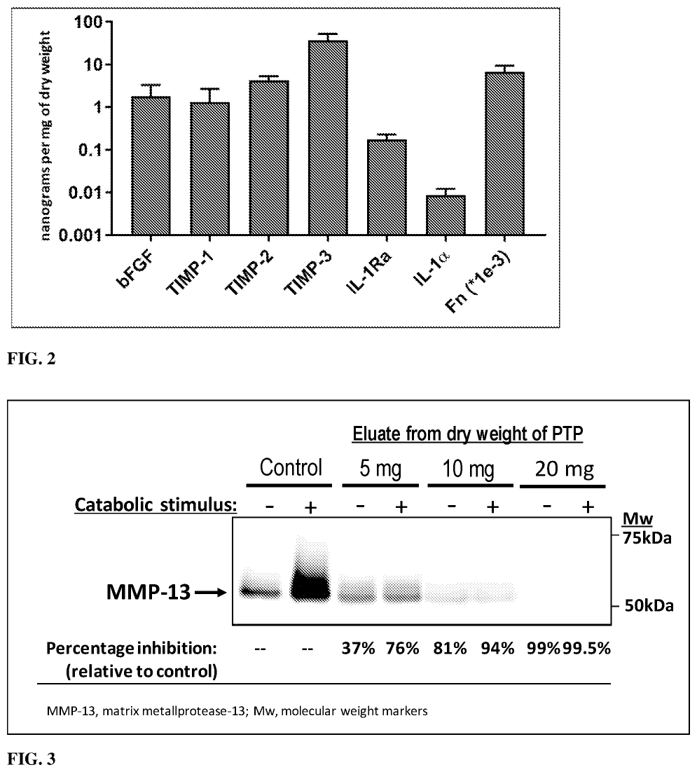

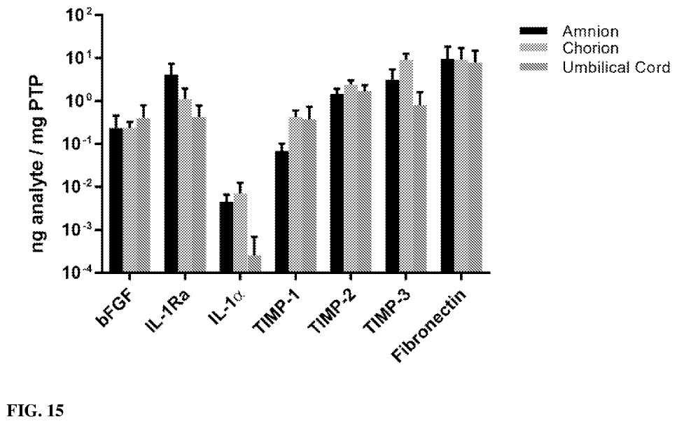

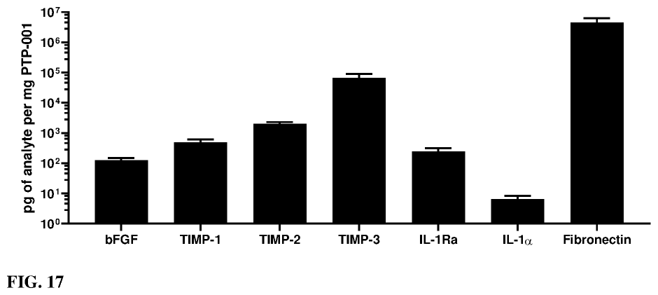

In general, the dehydrated PTP compositions of the invention contain from about 10 wt % to about 30 wt % amnion, about 30 wt % to about 75 wt % chorion and about 5 wt % to about 50 wt % umbilical cord tissue particulates. The inventors have found that the combination of these three placental tissues in these amounts provides significant therapeutic effects in the treatment of musculoskeletal disorders and pain associated with such disorders. The inventors have found that all three placental components contain beneficial endogenous growth factors and cytokines. In particular, each component has measurable levels of pro-anabolic (bFGF), anti-catabolic (TIMP-1,-2,-3), and anti-inflammatory (IL-1Ra) growth factors and cytokines; and consistently low levels of inflammatory factors (IL-1α) (See FIG. 15). The levels were found to be sufficiently comparable amongst all three components such that a formulation using a component ratio that maximizes the yield of the donor placenta is strongly preferred. The average preferred component ratio from 20 donor placentas is shown below in Table 1. The resulting levels of some of the key growth factors and cytokines were measured and can be found in FIG. 2 and FIG. 17.

Table 1 below shows the average amount of each tissue contained within PTP compositions of the invention (w/w) generated from 20 donor placentas.

TABLE 1
Average dry w/w % of each tissue component of PTP.

| N = 20 | AM | CH | UC |
|---|---|---|---|
| Min | 14% | 33% | 18% |
| Max | 35% | 58% | 49% |
| Average | 20% | 46% | 34% |
| Std Dev | 5.5% | 6.9% | 8.2% |

Additional components can be added to the PTP composition during processing as desired. In some embodiments, the lyophilized PTP composition can be mixed with dehydrated and similarly milled and sized collagen, fibrin, or HA, for example.

In another aspect of the invention, the dehydrated PTP composition can be resuspended in a suitable solution, buffer, or excipient, preferably at point of care. Exemplary solutions include but are not limited to normal saline (0.9% sodium chloride), a physiological salt solution (phosphate buffered saline; PBS), Dulbecco's Modified Eagle Solution (DMEM), water, any autologous preparation (such as platelet rich plasma (PRP), bone marrow aspirate concentrate (BMAC), stromal vascular fraction (SVF)), corticosteroid, a solution containing HA or anti-inflammatory agents, and balanced salt solution (BSS). Additionally, a resuspended solution of PTP can be co-delivered with one or more solutions containing additional therapeutic agents. For example, the resuspended PTP solution may be co-delivered with an HA solution, a solution containing anti-inflammatory agent(s), or SVF, BMAC, or PRP for example, using a dual barrel syringe for example to simultaneously deliver the different solutions.

The concentration of the PTP can be varied as needed. In some procedures a more concentrated preparation is useful, whereas in other procedures, a solution with a lower concentration of PTP is useful. In various embodiments of the invention, additional compounds or components can added to the composition. Exemplary compounds that can be added to the resuspended formulation include but are not limited to pH modifiers, buffers, collagen, HA, anti-inflammatories, surfactants, stabilizers, proteins, and the like. Antimicrobial agents such as antibiotics or anti-fungal agents may be added. Other substances can be added to the compositions to stabilize and/or preserve the compositions if needed. The material can be packaged and stored, for example, at room temperature, under refrigeration, or for example, at −20° C. or −80° C. prior to use.

Pharmaceutical compositions may be formulated in a conventional manner using one or more physiologically acceptable carriers including excipients and auxiliaries which facilitate processing of the PTP compositions into preparations which can be used pharmaceutically. Formulation is dependent upon the desired route of administration. Any of the well-known techniques, carriers, and excipients may be used as suitable and as understood in the art. A summary of pharmaceutical compositions described herein may be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins; 1999), herein incorporated by reference in their entirety.

In certain embodiments, the compositions include a pharmaceutically acceptable diluent(s), excipient(s), or carrier(s). In addition, the resuspended PTP preparations and PTP compositions described herein can be administered as pharmaceutical compositions in which the PTP compositions are mixed with other active ingredients, as in combination therapy. In some embodiments, the pharmaceutical compositions may include other medicinal or pharmaceutical agents, carriers, adjuvants, such as preserving, stabilizing, wetting or emulsifying agents, solution promoters, salts for regulating the osmotic pressure, and/or buffers. In addition, the pharmaceutical compositions can also contain other therapeutically valuable substances

In certain embodiments, resuspension of the dehydrated PTP to form a homogenous mixture generally does not require more than manual agitation of the vial containing the PTP. In such embodiments, the dehydrated PTP is preferably resuspended within one minute or less with manual agitation. Preferably, the resuspended PTP is suitable for injection or extrusion from a syringe. Resuspended formulations may include suspensions, pastes and putties that are extrudable from a syringe. For intra-articular injection, for example, the resuspended product is preferably readily deliverable through a 22 gauge needle.

The inventors have noted that the case and time of resuspension of dehydrated PTP may be affected by a number of factors, since dehydrated PTP particles may clump upon wetting and become difficult to disperse into a homogenous mixture. Such factors include resuspension technique, resuspension solution, design and composition of the vial, lyophilization method, irradiation level, presence of additives to improve wettability, and means of agitation. Any of these factors or combination of these and/or other factors known in the art may be employed to enhance the case of resuspension of the PTP compositions. For example, in one embodiment, e-beam irradiation is used to improve resuspension characteristics. In another embodiment, a surfactant or bulking agent is added to PTP prior to lyophilization that reduces the likelihood of particle clumping. In another embodiment, beads, such as glass beads or stainless steel beads may be added into the vial prior to lyophilization that can subsequently be used to agitate the PTP upon resuspension and break up any clumps that have formed. The beads are sized to be too large to be drawn up into an appropriately sized syringe for delivery of the PTP suspension and are not intended for injection or implantation.

In certain embodiments, dehydrated PTP compositions may be resuspended for injection at the time of point of care or may be mixed with an excipient and stored at 2 to 8° C., for example, for later use. For example, the compositions may be resuspended in a solution such as sterile sodium chloride solution, for example 0.9% sodium chloride (hereinafter referred to as “saline”). The resuspended PTP compositions generally contain about 25 mg PTP as a minimum therapeutically effective amount, preferably about 100 to about 500 mg, or from 150 to 300 mg dehydrated PTP, preferably 175 to 250 mg dehydrated/lyophilized tissue, such as 200 mg PTP particulates and from 1 to 10 mL of a solution, such as 6 mL, preferably 4 mL solution, such as saline, e.g., 0.9% sodium chloride. A preferred dosage unit is 50 mg/mL, however, the amount of PTP and solution may be varied as needed.

The PTP compositions may be resuspended for injection to treat osteoarthritis and the pain and dysfunction associated with the disease and may delay its progression and decrease pain associated therewith. The resuspended PTP compositions may also be administered by injection for the treatment of other orthopedic diseases and conditions, including but not limited to, degenerative disc disease, tendonitis, plantar fasciitis, cartilage deficits or damage, trauma, and soft tissue injuries and the pain associated therewith. Additionally, the resuspended PTP compositions may be administered by injection or localized placement to prevent or reduce scarring or inflammation for example. The resuspended PTP compositions also may be administered by injection or localized placement for protection of cartilage and/or for treatment of pain, such as synovial pain associated with OA for example.

Typically, the resuspended PTP compositions are administered directly to a target site (e.g., joints, surgical site, tendon). The administration of PTP formulations via intra-articular route and direct injection into a tendon, for example, are well-known in the art. Administration can also be parenteral (e.g., subcutaneous). Other methods of delivery, e.g., liposomal delivery, diffusion from a device impregnated with the rehydrated composition, and microemulsion-based transdermal delivery in pharmaceutical applications, are known in the art.

Alternatively, the PTP composition can be applied to a target site, e.g., a joint or tendon in a dry form, by ‘sprinkling’ a dosage of the dry PTP composition onto the target site. In another embodiment, a PTP composition, following rehydration to have a paste-like consistency, can be also be applied directly to a target site.

The PTP compositions described herein can be administered for prophylactic and/or therapeutic treatments. A “therapeutic amount” or “therapeutically effective amount” administered to an individual suffering from a disease or condition is an amount sufficient to cure or at least partially decrease one or more symptoms of the disease or condition. Therapeutic amounts will depend on several factors including severity and course of the disease or condition, previous therapy, the patient's health status, age, weight, and response to the drugs. It is considered well within the skill of the art for one to determine therapeutically effective amounts by routine experimentation (including, but not limited to, a dose escalation clinical trial).

For prophylactic treatments, the PTP compositions described herein may be administered to a patient susceptible to or at risk of a particular disease, disorder or condition. As with a therapeutic dose, a “prophylactically effective amount or dose” will depend on the patient's state of health, age, weight, and the like. It is considered well within the skill of the art for one to determine such prophylactically effective amounts by routine experimentation (e.g., a dose escalation clinical trial).

The dosages for the PTP compositions described herein are from about 25 to 500 mg/joint or tendon or other target site, for example 25 to about 50 mg, from 25 to 200 mg, from about 50 to 150 mg, from about 100 to about 200 mg, from 150 to 300 mg, from 200 to 400 mg, or from 300 to 500 mg per joint or tendon or other target site, or any range between, conveniently administered in single dose, optionally with follow up doses administered later in time, for example.

The inventors have found that compositions comprising the combination of AM, CM and UC in the specified ranges of amounts as described herein provide enhanced therapeutic effect at specific dose ranges in the treatment of OA. In general, an effective minimum dose of the PTP compositions of the invention for the treatment of pain, such as joint pain associated with OA, is a dosage amount of PTP greater than 25 mg, such as 50 mg, 100 mg, 150 mg, 200 mg, for example. A dosage regimen for the treatment of pain associated with musculoskeletal disorder may include intra-articular injection of 50 mg to 200 mg PTP, optionally followed by a repeat dosage via intra-articular injection within one week to six months, as needed. The treatment regimen may include further repeat dosages as needed. Single dose administration of greater than 25 mg, such as 50 mg, 100 mg, 150 mg, 200 mg of the PTP compositions of the invention results in significant relief of pain associated with OA, while repeat dosage administration of the PTP compositions provides a prolonged and significant relief of pain associated with OA.

For the treatment of non-pain related symptoms of musculoskeletal disorders, such as prevention or inhibition of the disorder per se, or for the treatment of surgical sites, a higher dosage amount than used for the treatment of pain is generally used. Typically, a dosage amount of from about 100 mg to 300 mg PTP, preferably 100 to 250 mg PTP, more preferably 100 to 200 mg PTP, is administered via injection, e.g., into the affected tissue, joint, or surgical site. In certain embodiments, the desired dose may be administered as a single dose or as divided doses administered simultaneously (or over a short period of time). Alternatively, staggered doses may be administered at appropriate intervals, for example as two or more doses administered one week to six months apart, such as two weeks to three months apart, two weeks to one month apart, etc., and optionally followed by one or more repeat doses as needed. Preferably, for the treatment of musculoskeletal disorders such as OA, at least two doses, e.g., each of about 100-200 mg, are administered two to four weeks apart. Further doses of the PTP composition are administered as needed.

The foregoing ranges and timing of doses are merely suggestive, as the number of variables in regard to an individual treatment regime is large. The timing of administration and amount of each dose may vary, depending on several factors including severity and course of the disease or condition, previous therapy, the patient's health status, age, weight, and response to the drugs. It is considered well within the skill of the art for one to determine a therapeutically effective dosing.

The pharmaceutical compositions described herein may be in unit dosage forms suitable for single administration of dosages. In unit dosage form, the formulation is divided into unit doses containing appropriate quantities of the PTP composition, e.g., 100 mg or 200 mg PTP. The unit dosage may be in the form of a package containing discrete quantities of the formulation. A non-limiting example includes lyophilized PTP powder in vials or ampules.

In another aspect of the technology, kits containing one or more aliquots of sterilized, dehydrated PTP powder with or without additional components and a separate and appropriate amount of solution for resuspending the PTP, e.g., 0.9% saline are provided. The kits may include an appropriate device for delivery of the PTP preparation, e.g., a vial adapter and/or a syringe and appropriately sized needle. The kits may also include instructions for resuspension and administration.

Studies conducted by the inventors have demonstrated that the PTP compositions of the invention contain a combination of beneficial anti-inflammatory, anti-catabolic and pro-anabolic proteins and growth factors. The release of such factors from the PTP compositions can, among other actions, reproducibly inhibit the production of one the principal collagenases, MMP-13, expressed by human articular cartilage cells (chondrocytes) that have been isolated in culture from donor arthritic cartilage.

The compositions and methods described herein are described in further detail in the following examples. These examples are provided by way of illustration and are not intended to limit the invention in any way.

### EXAMPLES

### Example 1

### PTP Composition Preparation

Each of 11 lots of the PTP compositions was derived from placental tissue from a single donor. Prior to processing, donated placental tissue was aseptically recovered from a live Caesarian birth, transferred to an FDA-registered tissue establishment, placed into quarantine and donor information was entered into a database for tissue tracking.

Donor placental tissue processing was initiated within 72 hours from the time of recovery. Prior to processing, tissue was inspected and an incoming bioburden sample was collected. The donated placental tissue was then separated (i.e., amnion, chorion, and umbilical cord, which was measured at time of acquisition and later dissected to remove blood vessels), cleaned to remove blood clots, rinsed with phosphate buffered saline (3-5 minutes per rinse at room temperature), cut into smaller pieces, e.g., 4 cm strips and lyophilized. Following lyophilization, the tissue was cut again to facilitate cryogenic milling. The individual milled placental tissues were weighed, combined, and sieved between 20 and 150 μm sieves to produce placental tissue particulate. The tissues were combined in ratios that maximized the yield of the PTP composition. The dry weight percentages of each placental tissue in the combined PTP are shown in Table 1. This placental tissue particulate was then filled into single-use Type I glass vials (aliquoted by weight) that were lyophilized a final time, stoppered and sealed. Each vial of PTP was then packaged in a foil pouch and sealed prior to terminal sterilization by electron beam irradiation.

### Example 2

### Protein Analyses of Growth Factor/Cytokine Content

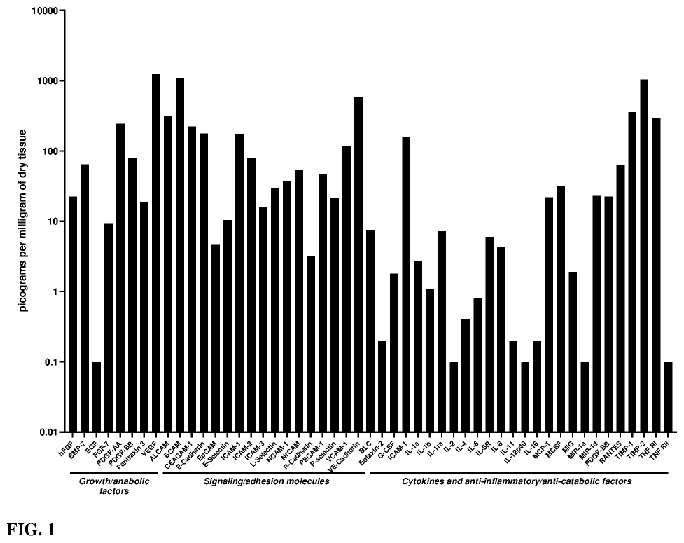

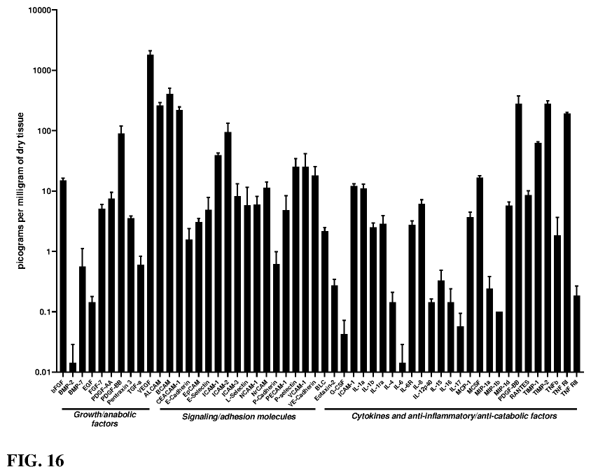

Protein multiplex analyses were performed on PTP samples using a custom Quantibody® Array to assess for the presence of unique pro-anabolic, inflammatory and anti-catabolic/anti-inflammatory growth factors and cytokines. Samples of PTP were suspended in an enzymatic solution (0.2% w/v collagenase type I dissolved in phosphate buffered saline; 0.5 ml per 10 mg dry weight of PTP) and incubated at 37° C. for 24 hours. After centrifugation the supernatants were collected and frozen at −80° C. and transferred on dry ice to RayBiotech® Norcross, GA for Quantibody® multiplex analysis. Multiplex data derived from seven individual donors and samples yielded readily detectable levels of a range of pro-anabolic, anti-catabolic, and anti-inflammatory growth factors and cytokines (see FIG. 1 and FIG. 16). Data from a single donor are displayed in FIG. 1 while data from seven donors are displayed in FIG. 16.

Next, a select panel of therapeutically relevant, representative growth factors and cytokines were chosen to perform quantitative analysis using enzyme-linked immunosorbent assays (ELISAs) of samples prepared as described above for the Quantibody® multiplex analyses. For example, detection of basic fibroblast growth factor (bFGF) can be assessed as an indicator of pro-anabolic function, tissue inhibitor of metalloproteases (TIMP)-1, -2 and -3 levels can be determined to demonstrate the presence of therapeutically important anti-catabolic factors, and interleukin-1 receptor antagonist (IL-1Ra) levels can be measured to represent a key anti-inflammatory marker. In addition, ELISAs were performed for the extracellular matrix (ECM) molecule fibronectin. The pro-inflammatory factor IL-la was also measured by ELISA to ensure consistent but low levels of such activity in the PTP preparations (see FIG. 2).

FIG. 2 shows the results of an analysis of individual donors (n=7 donors) which demonstrates high levels of pro-anabolic (bFGF), anti-catabolic (TIMP-1,-2,-3), and anti-inflammatory (IL-1Ra) growth factors and cytokines; and low levels of inflammatory factors (IL-la). The ECM molecule fibronectin was also readily detected (n=6 donors for this analysis). Data are presented as mean and standard deviation with single determination per donor.

Additionally, all of the ELISA methods were externally validated by a third-party contract development and manufacturing organization in accordance with ICH Q2 (R1). Samples from 12 donors were tested using these validated assays. Validation activities ensure specificity of the assay, accuracy of the assay, precision between assay kits, and precision between multiple assay operators. FIG. 17 shows the results of the analysis using validated ELISAs (n=12 donors, data: mean+SD). These results are consistent with FIG. 2—high levels of pro-anabolic, anti-catabolic, and anti-inflammatory growth factors and cytokines with low levels of inflammatory factors.

### Example 3

### In Vitro Cell-Based Analyses

Human chondrocyte bioassay: Articular chondrocytes from OA patients are used as a relevant model to test the effects of the PTP compositions on human osteoarthritic cartilage. Chondrocytes isolated from excised cartilage from OA patients undergoing knee replacement surgery were cultured as cell monolayers under basal or catabolic (e.g., addition of a matrikine fragment of fibronectin) conditions, and in the presence of eluates from PTP preparations (see below). Following the culture period, key outcome measures of cell viability, indicators of anabolic processes, and ECM catabolismwere assessed.

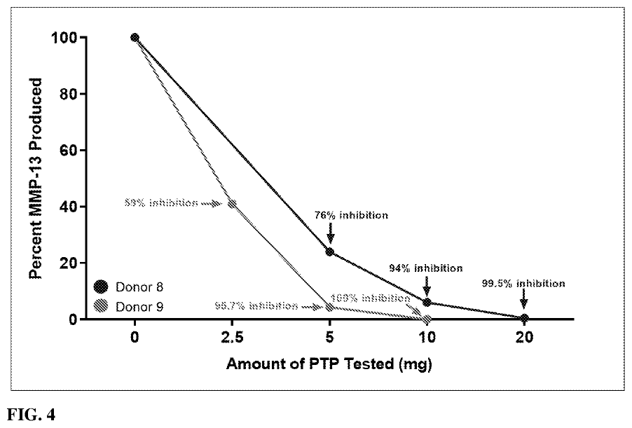

Eluates from PTP preparations were prepared by incubating tissue samples in serum-free culture media for 48 hours. Chondrocyte pre-culture media was removed and replaced with the PTP eluates followed by a further 48 hour culture period until basal or catabolic conditions. Media samples were then analyzed by Western immunoblotting for the presence of MMP-13 (collagenase-3) protein levels. Cell monolayers were assessed for viability using LIVE/DEAD reagents (Molecular Probes). Protein bands detected by Western blotting were quantified using NIH ImageJ software. As shown in FIGS. 3 and 4, there was a strong induction of MMP-13 expression levels by the fibronectin matrikine (e.g., catabolic stimulus). At the doses tested, treatment of the chondrocyte cultures with PTP eluates resulted in a strong, and apparent dose-dependent inhibition of MMP-13 production, both in the absence or presence of the catabolic stimulus. These initial results indicate that PTP may also play a protective role in OA joints by reducing inflammation-associated MMP production, and inhibiting MMP-mediated extracellular matrix degradation.

### Example 4

### Surgically-Induced OA Model in Rats.

An in vivo rat OA model (N=12 animals per group) was conducted wherein unilateral OA was induced by surgical destabilization of the medial meniscus (DMM). At 2 weeks post-surgery, animals received an intra-articular injection (50 μL) of saline, PTP suspended in saline (50 μL; 1.25 or 2.5 mg/joint; equivalent to 100 mg and 200 mg human dose, respectively), or steroid in saline (0.06 mg/joint). Additional animal groups received a second injection of PTP (1.25 or 2.5 mg/joint) at 4 weeks post-surgery. Pain measurements assessing hindlimb weight-bearing (incapacitance testing) or mechanical allodynia (Von Frey analysis) were taken weekly for 6 weeks post-initial treatment. At the end of the study, knee joints were sectioned for histopathologic assessment to include: cartilage damage/loss, synovial inflammation and fibrosis, bone/calcified cartilage resorption, and osteophyte/chondrocyte proliferation.

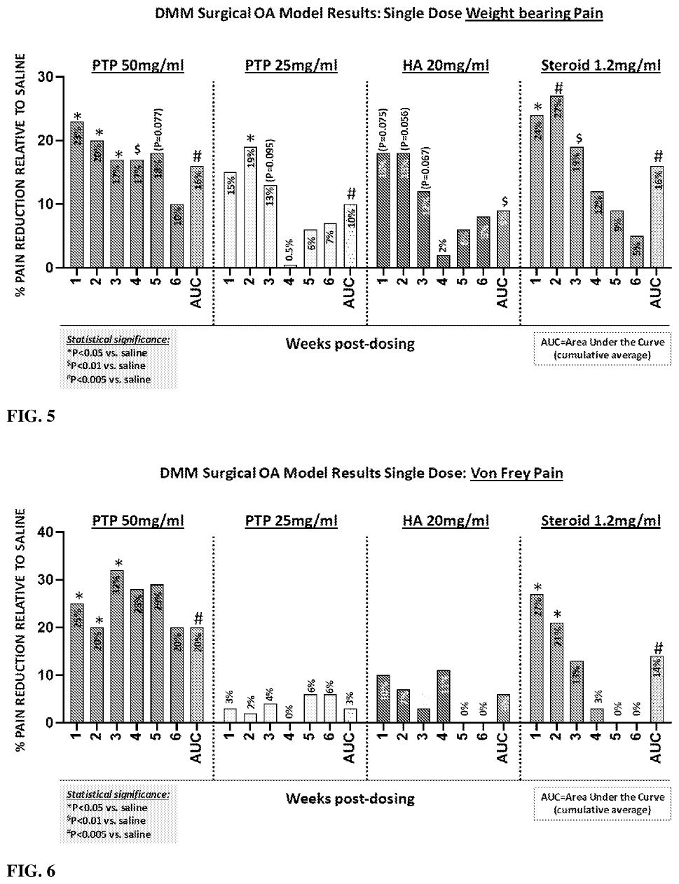

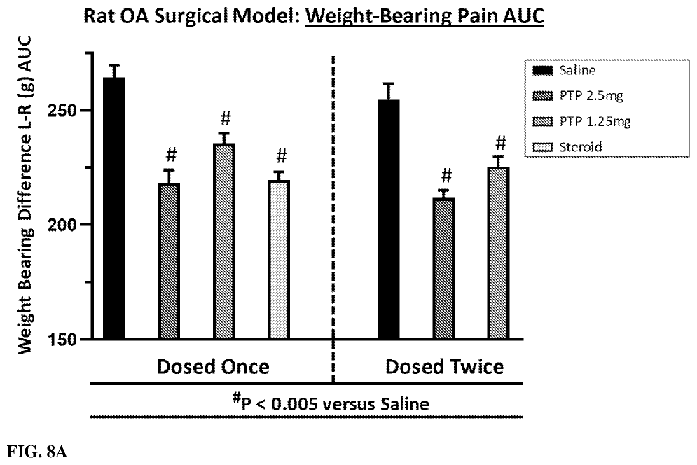

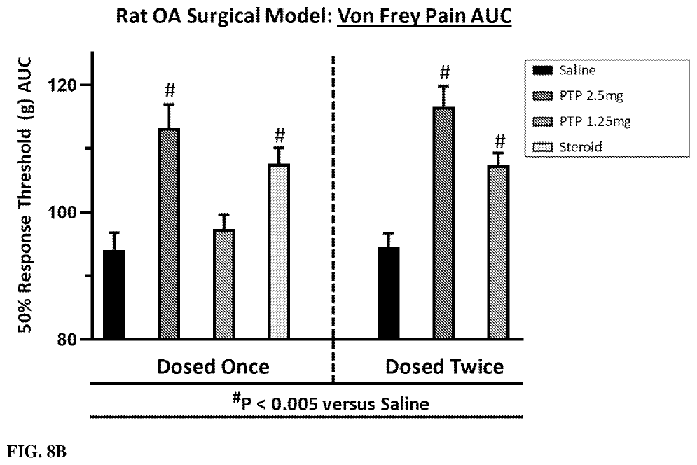

Animals which received a single 2.5 mg/joint dose of PTP comprising about 25 wt % amnion, about 45% wt % chorion, and about 30 wt % umbilical cord, a PTP composition of the invention, demonstrated a significant reduction in weight-bearing pain (hindlimb weight imbalance) relative to saline treated animals as early as 1 week after dosing (P<0.05; see FIG. 5a). Pain reduction for the 2.5 mg/joint dose of PTP was maintained throughout the study, with a cumulative average (area under the curve; AUC) of 16% pain reduction (P<0.005 vs. saline treatment) over 6 weeks post-dosing (FIG. 5 and FIG. 8A). An apparent dose response was observed for animals receiving 25 mg/mL (1.25 mg/joint) PTP, with a cumulative average of 10% pain reduction over 6 weeks post-dosing (P<0.005 vs. saline). In comparison, while animals which received a single injection of corticosteroid (1.2 mg/mL triamcinolone) displayed significant pain reduction at early timepoints (weeks 1-3), this effect waned dramatically at weeks 4-6. A 16% cumulative average pain reduction over 6 weeks (P<0.005 vs. saline) was observed for corticosteroid treated animals.

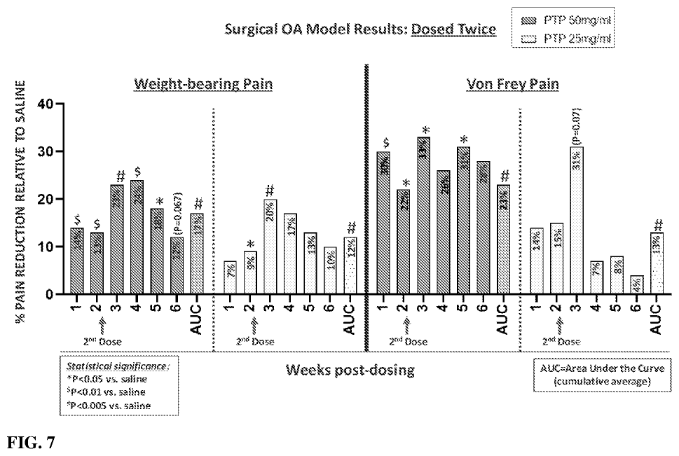

The results of Von Frey pain testing on these same animals yielded similar overall trends (see FIG. 6). For the 2.5 mg/joint dose of PTP, substantial pain reduction levels of 20%-32% were observed at each weekly timepoint post-dosing, with a cumulative average of 20% pain reduction over 6 weeks (P<0.005 vs. saline; FIG. 6 and FIG. 8B). The 1.25 mg/joint PTP dose, and corticosteroid, displayed a markedly attenuated response using this pain metric.

Results from separate groups of animals which received 2 doses of PTP (or saline), 2 weeks apart, are shown in FIG. 7. Weight-bearing and Von Frey pain results for animals dosed twice with 2.5 mg/joint PTP were similar to those observed for the animals which were only dosed once. For the animals that received 2 doses of 1.25 mg/joint PTP, the overall magnitude of effect was greater than that observed for animals that had received a single dose, with cumulative averages of 12% (P<0.005 vs. saline) and 13% (P<0.005 vs. saline) pain reduction over 6 weeks for weight-bearing and Von Frey pain assessments, respectively (see FIGS. 7, 8A, and 8B).

Cartilage damage was assessed and scored as follows:
Cartilage Degeneration Score: Sum of 3 zones (outer, middle, inner) scored from 0-5, where 0=no degeneration; 5=severe degeneration (>50% loss of cells with substantial matrix loss).
Total Joint Score: Sum of cartilage degeneration score and osteophyte score (osteophytes scored by size, where 0=less than 0.2 mm; 5=greater than 0.6 mm).
Width of Severe Lesions: Determined for areas where cartilage lesions penetrate >50% of the tissue depth.
Depth Ratio: Determined by dividing the lesion depth by the total cartilage depth. This measurement is the most critical analysis of any type of microscopic change present.

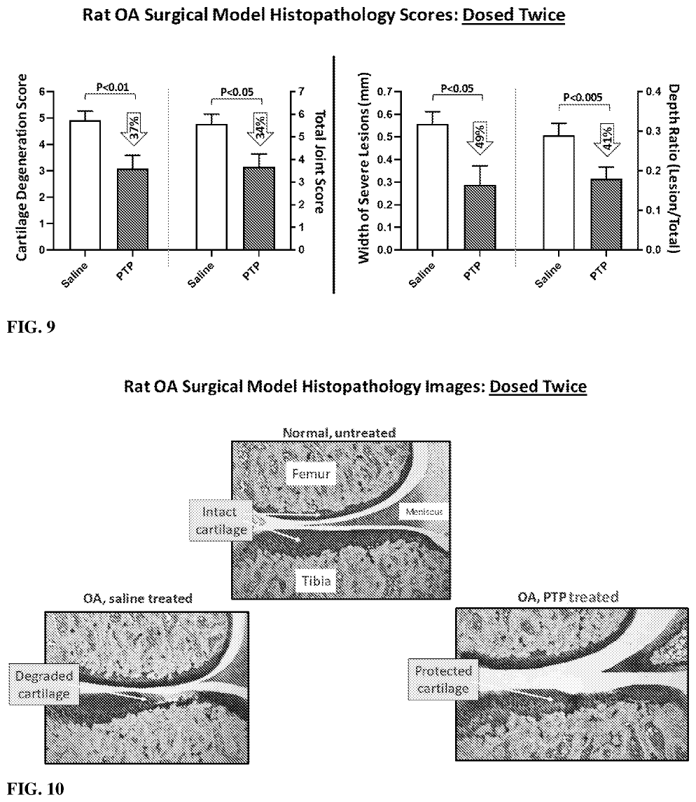

Results from the separate groups of animals are shown in FIGS. 9 and 10. The following is a summary of the histopathology effects observed:
Animals which received 2 treatments of PTP high dose (50 mg/ml), relative to saline treatment, had significantly reduced cartilage degeneration scores (37% reduction; P<0.01), total joint scores (34% reduction; P<0.05), width of severe lesions (49% reduction; P<0.05), and cartilage lesion depth ratios (41% reduction; P<0.005).

### Example 5

### Collagenase-Induced Tendinitis/Tendinopathy Model in Rats.

An in vivo rat tendinitis model (N=10 animals/group) was conducted wherein unilateral tendinitis was induced by direct injection (30 μL) of collagenase (C-6885, 0.3 mg in PBS; Sigma, St. Louis, MO) into the Achilles tendon on Day 0 and Day 1 of the study. A separate group of animals served as a sham control (saline injection only) for histopathology analysis. On Day 5 of the study, animals which had received a collagenase injection were treated with an intra-tendon injection (30 μL) of saline, or PTP suspended in saline (30 μL; 1.5 mg/tendon), or steroid in saline (0.3 mg/tendon). Pain measurements assessing hindlimb weight-bearing (incapacitance testing) or mechanical allodynia (Von Frey analysis) were taken at 2, 4, 9, 16, 23, and 30 days post-treatment. At the end of the study, tendons (attached to bone of ankle after decalcification) were sectioned for histopathologic assessment to determine extent of tendon damage, inflammation, and character of tendon repair.

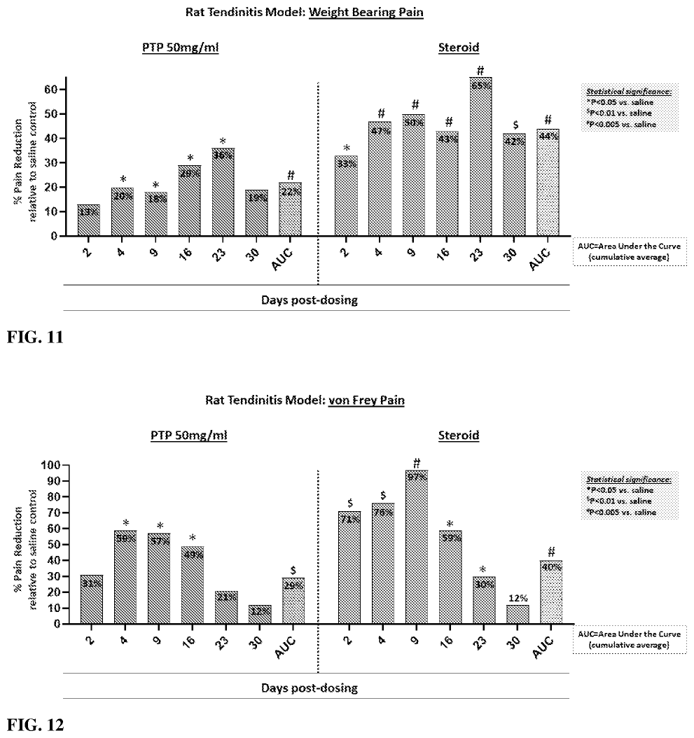

Animals which received a single 1.5 mg/tendon dose of PTP comprising about 25 wt % amnion, about 45% wt % chorion, and about 30 wt % umbilical cord, demonstrated a significant reduction in weight-bearing pain (hindlimb weight imbalance) relative to saline treated animals as early as 4 days after dosing (P<0.05; see FIG. 11) which was maintained over the course of the study with a cumulative average (AUC) of 22% pain reduction (P<0.005 vs. saline treatment). Animals which received a single injection of corticosteroid (1.0 mg/mL triamcinolone) also displayed significant pain reduction (FIG. 11).

The results of Von Frey pain testing on these same animals yielded similar overall trends, however with greater magnitudes of pain reduction relative to saline treated animals (see FIG. 12).

Histopathologic changes were assessed and scored as follows:
Tendon Damage Score: Scored from 0-5, where 0=normal/no damage; 5=severe focal or multifocal areas of damage (affecting >75% of tendon area).
Tendon Inflammation Score: Scored from 0-5, where 0=normal; 5=severe diffuse infiltration of inflammatory cells in tendon or tendon sheath, with severe extension into peripheral adipose tissue (affects >75% of adipose tissue, dense infiltration).
Tendon Repair Score: Scored from 0-6 (0=No repair/proliferative tissue, due to no damage ever present), where 1=minimal repair with minimal collagen fibril deposition and vascularization; 6=Normal as a result of total or near total repair with well aligned connective tissue bundles.

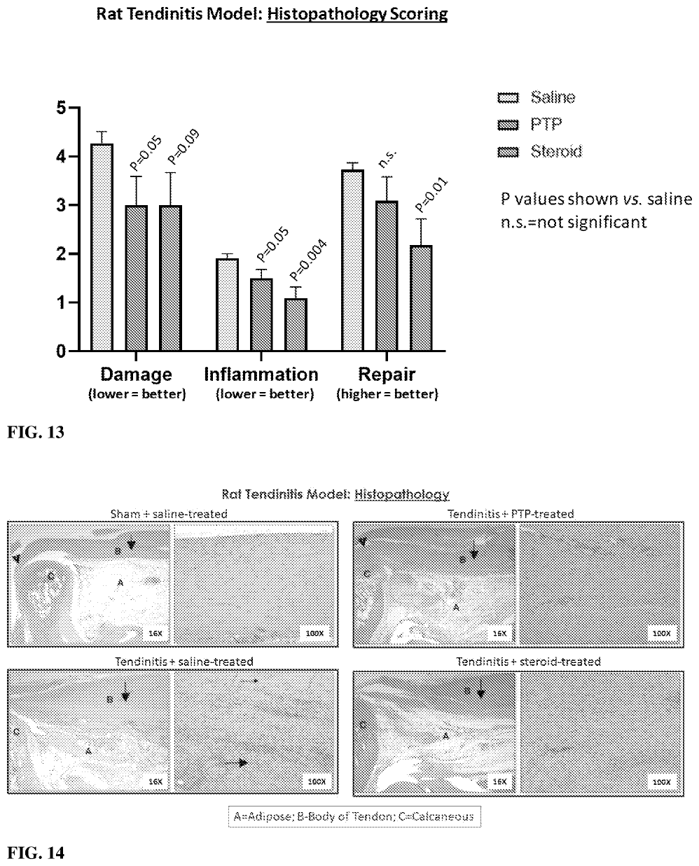

Results from the separate groups of animals are shown in FIGS. 13 and 14. The following is a summary of the histopathology effects observed:

Animals which received treatment with PTP (50 mg/ml), relative to saline treatment, had significantly reduced tendon damage and inflammation scores (P=0.05). PTP treated animals displayed a substantial level of tendon repair. In comparison, while steroid treated animals also had significantly reduced inflammation scores relative to saline treatment, tendon repair was significantly reduced (FIG. 13).

### Example 6: Preservation of Endogenous Growth Factor Content

Various processing pathways were studied to determine methods which best preserve endogenous growth factor content (See Table 2).

TABLE 2
Processing Conditions for Placental Tissue Particulate

| Process | Cleaning/Disinfection | 1^{st} | Freezer | 2^{nd} | E- |
|---|---|---|---|---|---|
| ID | Process | Lyophilization | milling | Lyophilization | beam |
| A | Rinse only (no disinfection) | ✓ | | | |
| B | Rinse only (no disinfection) | ✓ | ✓ | ✓ | |
| C | Rinse only (no disinfection) | ✓ | ✓ | ✓ | ✓ |
| D | Chemical disinfection | ✓ | ✓ | ✓ | |
| | process 1 | | | | |
| E | Chemical disinfection | ✓ | ✓ | ✓ | |
| | process 2 | | | | |

E-beam = electron beam irradiation

It was found that processing conditions that include rinsing only and no additional chemical disinfection process were better at preserving the amount and bioactivity of the bioactive components of the PTP tissues (e.g., bFGF, IL-1Ra, IL-1α, TIMP-1, TIMP-2, TIMP-3, and fibronectin) compared to either chemical disinfection process. It was also found that E-beam irradiation of the PTP composition as a terminal sterilization step did not have a significant effect on bioactivity or content of the bioactive components.

1.-28. (canceled)

29. A method for preparing a particulate composition obtained from mammalian placental tissue comprising the steps of:
(1) separating AM, CM and UC from tissue;
(2) cutting the AM, CM and UC into multiple pieces to obtain separate AM, CM and UC tissue pieces and lyophilizing the pieces;
(3) cryomilling the AM, CM and UC tissue pieces separately to obtain particulates having a particle size of 20 to about 150 microns;
(4) combining a predetermined amount of each of the AM, CM and UC milled tissue to obtain a mixture comprising from about 10 wt % to about 30 wt % AM particulates, about 30 wt % to about 75 wt % CM particulates and about 5 wt % to about 50 wt % UC particulates;
(5) lyophilizing the mixture; and
(6) sterilizing the particulates of step (5).

30. The method of claim 29, further comprising the step of reconstituting the particulates in a sufficient amount of a sterile aqueous solution to form a suspension of the particulates.

31. The method of claim 30, wherein the suspension comprises a dose of about 40 mg to about 200 mg of the particulate composition.

32. The method of claim 30, wherein the suspension is designed for intra-articular administration comprising a dose of about 100 mg to about 200 mg of the particulate composition

33. The method of claim 30, wherein the suspension is designed for intra-articular administration comprising a dose of about 200 mg of the particulate composition.

34. The method of claim 29, wherein the particulate composition further comprises an amount of each of bFGF, IL-1Ra, IL-1α, TIMP-1, TIMP-2, TIMP-3, and fibronectin.

35. The method of claim 29, wherein the particulate composition is used to treat a musculoskeletal disorder comprising osteoarthritis, degenerative disc disease, cartilage deficits, soft tissue orthopedic injuries, plantar fasciitis, tendonitis, and rotator cuff injury.

36. The method of claim 29, wherein the particulate composition is used to provide durable mitigation of pain symptoms for 6 months or longer.

37. The method of claim 29, wherein the particulate composition promotes clinically meaningful improvement in functional recovery and delays cartilage degeneration in chronic musculoskeletal disorders

38. The method of claim 29, wherein the particulate composition further comprises hyaluronic acid, hyaluronic acid derivatives, antibiotics, anti-inflammatoires, corticosteroids, surfactants, or anti-caking agents.

39. A method for treating a musculoskeletal disorder in a patient, comprising:
(a) administering by localized injection to a knee joint in the patient, a first dose of a therapeutically effective amount of a particulate composition comprising a mixture of placental tissue particulates (PTP), said PTP comprising about 10 wt % to about 30 wt % AM particulates, about 30 wt % to about 75 wt % CM particulates and about 5 wt % to about 50 wt % UC particulates;
(b) reducing pain as measured by a patient reported outcome score;
(c) increasing new tissue formation as measured by X-ray or MRI; and
(d) delaying progression of tissue damage as measured by X-ray or MRI, or delaying secondary intervention such as additional injections of the same therapy or other approved therapies or total joint replacement;
wherein the therapeutically effective amount of the particulate composition is at least 150 mg.

40. The method of claim 39, wherein the particulate composition is configured to have a half-life of at least 30 days after administration.

41. The method of claim 39, wherein the particulate composition is configured to be detectable in the joint at least 60 days after administration.

42. The method of claim 39, wherein reducing pain comprises more than an eight point difference in mean change from a baseline WOMAC pain score.

43. The method of claim 39, wherein a second dose into the same location of a therapeutically effective amount of the particulate composition is administered within about two weeks.

44. The method of claim 39, wherein the therapeutically effective amount is about 100 to 200 mg.

45. The method of claim 39, wherein the particulate composition comprises quantifiable amounts of each of bFGF, IL-1Ra, IL-1α, TIMP-1, TIMP-2, TIMP-3, and fibronectin.

46. A method for treating pain associated with musculoskeletal disorder in a subject, comprising administering a therapeutically effective amount of a particulate composition to a patient in need of treatment; wherein the composition comprises about 10 wt % to about 30 wt % AM particulates, about 30 wt % to about 75 wt % CM particulates and about 5 wt % to about 50 wt % UC particulates; and
wherein administering the therapeutically effective amount of the particulate composition causes at least a 25% inhibition of inflammation.
wherein the therapeutically effective amount is about 200 mg.

47. The method of claim 46, wherein administering the particulate composition is by a localized injection.

48. The method of claim 46, wherein the particulate composition comprises quantifiable amounts of each of bFGF, IL-1Ra, IL-1α, TIMP-1, TIMP-2, TIMP-3, and fibronectin.
